(19) United States
(12) Patent Application Publication
(10) Pub. No.: US 2010/0286472 A1
(43) Pub. Date: Nov. 11, 2010

(54) NEEDLE FOR SURGICAL THREADING AND METHOD FOR USING THE SAME

(75) Inventor: MENAHEM NEUMAN, Carmei-Yosef (IL)

Correspondence Address: DANIEL J SWIRSKY, 55 REUVEN ST., BEIT SHEMESH, 99544, IL

(73) Assignee: Ilana Neuman

(21) Appl. No.: 12/563,187

(22) Filed: Sep. 21, 2009

(30) Foreign Application Priority Data
Oct. 5, 2008 (IL) 194519

(51) Int. Cl. A61B 17/34; A61F 2/04

(52) U.S. Cl. 600/37; 606/185

(57) ABSTRACT

In one aspect, the present invention may be directed to a needle for surgical threading of a strap of an implant through a tissue, the needle comprising: a trap for trapping the strap to the needle while the needle may be at the accessible side of the tissue; a tip for threading the trapped strap from the accessible side to the opposing side; and a mechanism for releasing the trap, the mechanism driven from the accessible side of the tissue, thereby allowing return of the tip to the accessible side of the tissue while abandoning the strap at the threaded point, thus performing the threading from the accessible side of the tissue.

### CROSS-REFERENCE TO RELATED APPLICATIONS

The current application claims the benefit of and priority to Israel Patent Application No. 194519, filed Oct. 5, 2008, and incorporated herein by reference in its entirety.

### FIELD OF THE INVENTION

The present invention relates to the field of pelvic floor reconstruction. In particular, the present invention relates to the field of pelvic floor reconstruction using implants.

### BACKGROUND OF THE INVENTION

Pelvic organ prolapse (POP) is a common female problem that can have a profound impact on a woman's quality of life.

The organs in the pelvic cavity, uterus, vagina, bladder and rectum, are held in place by a web of muscles and connective tissues that act much like a hammock. When these muscles and tissues become weakened or damaged, one or more of the pelvic organs shift out of normal position and literally “fall” into the vagina.

Prolapse surgical reconstruction is performed through the vagina. During the procedure, the surgeon repositions the prolapsed organs, securing them to surrounding tissues and ligaments, and may use a synthetic non-absorbable polypropylene mesh implant.

However, the prior art surgical procedures penetrate the patient from several directions.

As well, they do not provide reliable anchoring of the mesh implant.

It is an object of the present invention to provide a reliable anchoring of the mesh implant.

Other objects and advantages of the invention will become apparent as the description proceeds.

### SUMMARY OF THE INVENTION

The following embodiments and aspects thereof are described and illustrated in conjunction with systems, tools methods, and so forth, which are meant to be merely illustrative, not limiting in scope.

In one aspect, the present invention may be directed to a needle for surgical threading of a strap of an implant through a tissue, the needle comprising:
a trap for trapping the strap to the needle, while the needle may be at the accessible side of the tissue;
a tip for threading the trapped strap from the accessible side to the opposing side; and
a mechanism for releasing the trap, the mechanism driven from the accessible side of the tissue,
thereby allowing return of the tip to the accessible side of the tissue while abandoning the strap at the threaded point, thus performing threading from the accessible side of the tissue.
   The trap may comprise:
a niche, for inserting an end of the strap of the implant; and
a rod, for grasping the end of the strap.
   The niche may be located near the tip.

The end of the strap may comprise a looped end for inserting the rod thereinto.

According to another embodiment the rod is capable of applying physical force on the end of the strap towards the limiting wall thereof in the niche.

The mechanism for releasing the trap may be manually driven.

The mechanism for releasing the trap may comprise a cable, driven from the accessible side of the tissue, for removing the rod from the end of the strap.

The needle may further comprise an arm for driving the mechanism, the arm located outside the surgical area.

In another aspect, the present invention is directed to an anterior implant comprising:
at least two first straps for threading thereof into the arcus tendineous fascia pelvic (ATFP) ligaments;
at least two second straps for threading thereof into the sacrospinous (SS) ligaments; and
a loop between the second straps for anchoring thereof to the cervix.

The anterior implant may be used for reconstructing the organs selected from the group including: prolapse of the urinary bladder, the colon, the small intestine.

The anterior implant may further comprise spaces for reducing weight of the implant.

In another aspect, the present invention is directed to a posterior implant comprising:
at least two straps for threading thereof into the sacrospinous (SS) ligaments;
a first loop between the straps for anchoring thereof to the cervix; and
a second loop at the side opposing the straps, the second loop for anchoring thereof to the perineal body.

The posterior implant may be used for reconstructing the organs selected from the group including: the colon, the small intestine, the uterus.

The posterior implant may further comprise spaces for reducing weight of the implant.

In another aspect, the present invention is directed to a method for using a needle to thread a strap through a surface, the method comprising the steps of:
trapping an end of the strap while the needle is at the accessible side of the surface tissue;
threading the needle, together with the trapped strap, through the surface, from the accessible side of the surface;
releasing the trap, such that the driving of release is from the accessible side; and
returning the needle to the accessible side while abandoning the strap at the threaded point,
thereby performing threading from the accessible side.

The trapping of the end of the strap may comprise the steps of:
inserting the end of the strap into a niche; and
grasping the end of the strap.

Grasping of the end of the strap may comprise the step of inserting a rod of the needle into a looped end of the strap.

According to another embodiment grasping of the end of the strap may comprise the step of applying physical force on the end of the strap towards the limiting wall thereof in the niche.

Releasing the trap may comprise the step of removing the rod from the end of the strap.

In another aspect, the present invention is directed to a method for installing an anterior implant, the method comprising the steps of:
threading at least two first straps of the implant into the arcus tendineous fascia pelvic (ATFP) ligaments;
threading at least two second straps of the implant into the sacrospinous (SS) ligaments; and
anchoring a loop between the second straps to the cervix.

In another aspect, the present invention is directed to a method for installing a posterior implant, the method comprising the steps of:
threading at least two straps of the implant into the sacrospinous (SS) ligaments;
anchoring a first loop between the straps, to the cervix; and
anchoring a second loop at the side opposing the straps, to the perineal body.

In addition to the exemplary aspects and embodiments described above, further aspects and embodiments will become apparent by reference to the figures and by study of the following detailed description.

### BRIEF DESCRIPTION OF THE DRAWINGS

The objects and features of the present invention will become apparent from the following detailed description considered in conjunction with the accompanying drawings, in which:

FIG. 1 illustrates an anterior implant according to one embodiment of the present invention.

FIG. 2 illustrates an anterior view of the pelvic area before installing the anterior implant.

FIG. 3 illustrates the view of FIG. 2 after installing the anterior implant.

FIG. 4 illustrates a posterior implant according to one embodiment of the present invention.

FIG. 5 illustrates the view of FIG. 2 after installing the posterior implant.

FIG. 6 illustrates the head of a needle for threading the straps of the implants, according to one embodiment of the present invention.

FIG. 7 illustrates the first step of threading the straps of the implants, using the needle of FIG. 6.

FIG. 8 illustrates the second step of threading the straps of the implants, using the needle of FIG. 6.

FIG. 9 illustrates the third step of threading the straps of the implants, using the needle of FIG. 6.

FIG. 10 illustrates the fourth step of threading the straps of the implants, using the needle of FIG. 6.

FIG. 11 illustrates the needle of FIG. 6 and its operation.

FIG. 12 illustrates the operation of the needle of FIG. 6 from the aspect of the surgeon's access to the pelvic area.

FIG. 13 illustrates the operation of the needle of FIG. 6 in the aspect of FIG. 12, to another ligament.

It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, are merely intended to conceptually illustrate the structures and procedures described herein. Reference numerals may be repeated among the figures in order to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In some instances, well-known methods, procedures, components and circuits have not been described in detail, for the sake of brevity.

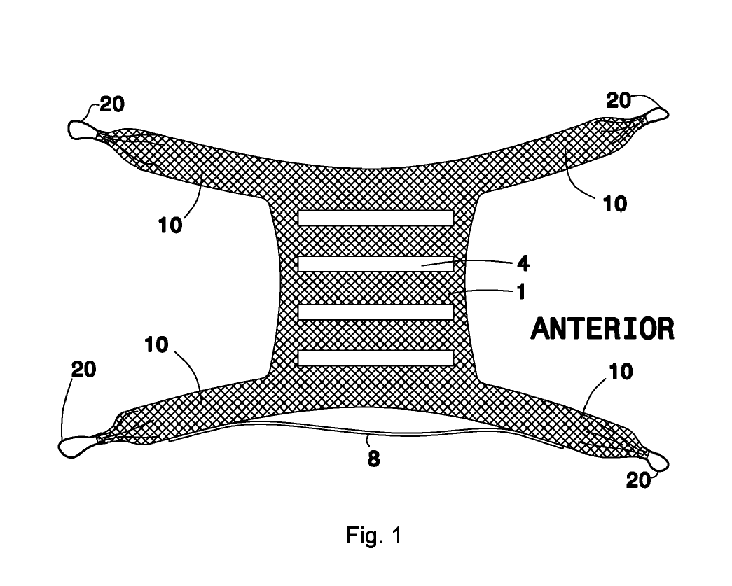

FIG. 1 illustrates an anterior implant according to one embodiment of the present invention.

Anterior implant 1 includes four straps 10, each ending with a looped end 20. Anterior implant 1 may also include a loop 8 between two interior straps 10.

Anterior implant 1 may include spaces 4 for reducing the weight thereof.

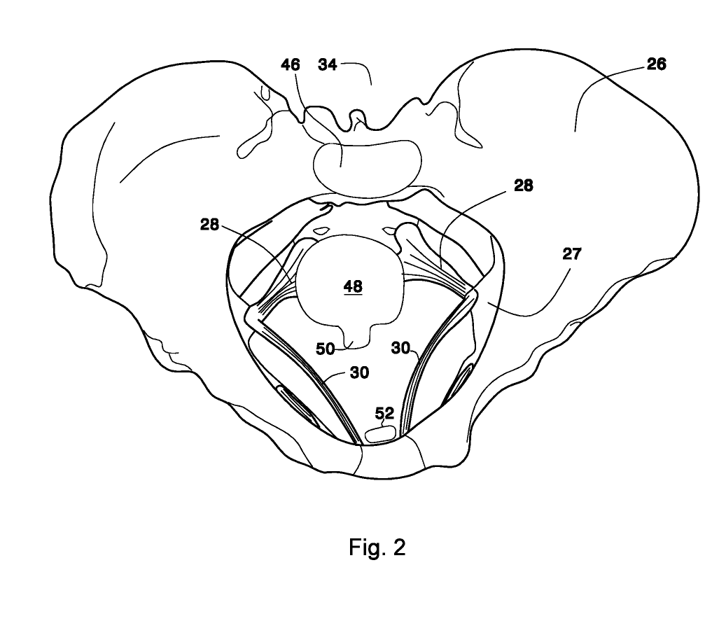

FIG. 2 illustrates an anterior view of the pelvic area before installing the anterior implant.

The anterior view of pelvic area 34 refers to the side allowing surgical access through the patient's vaginal opening.

Denoted bones are the ischial spine 27 extending from the posterior border of the ischium 26, and the sacrum 46.

Also shown are the perineal body 52, and the cervix 50 extending from the uterus (womb) 48.

Pelvic area 34 includes two ligaments of arcus tendineous fascia pelvic (ATFP) 30 and two ligaments of sacrospinous (SS) 28.

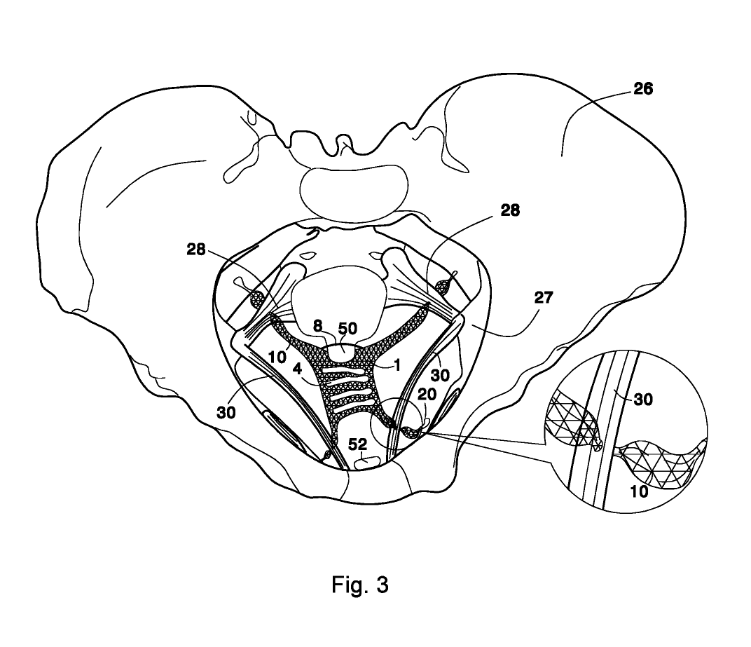

FIG. 3 illustrates the view of FIG. 2 after installing the anterior implant.

Anterior implant 1 is used for reconstructing the anterior pelvic floor, including prolapse of the urinary bladder and/or the colon and the small intestine.

Two straps 10 of anterior implant 1 are threaded into two ATFP ligaments 30, and the other two straps 10 are inserted into two SS ligaments 28.

Loop 8 may be sutured to cervix 50 for improving strength and security of the anchoring of anterior implant 1.

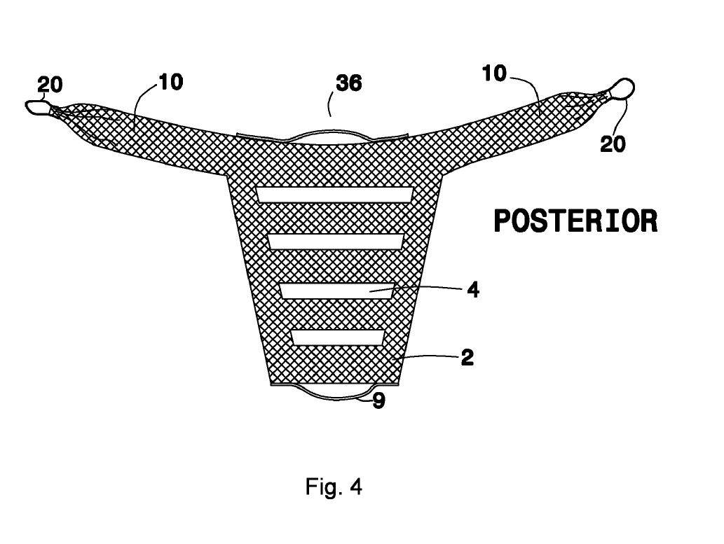

FIG. 4 illustrates a posterior implant according to one embodiment of the present invention.

Posterior implant 2 includes two straps 10, each ending with a looped end 20. Posterior implant 2 may include a loop 36 between two interior straps 10, and another loop 9 at the opposing side.

Posterior implant 2 may include spaces 4 for reducing the weight thereof.

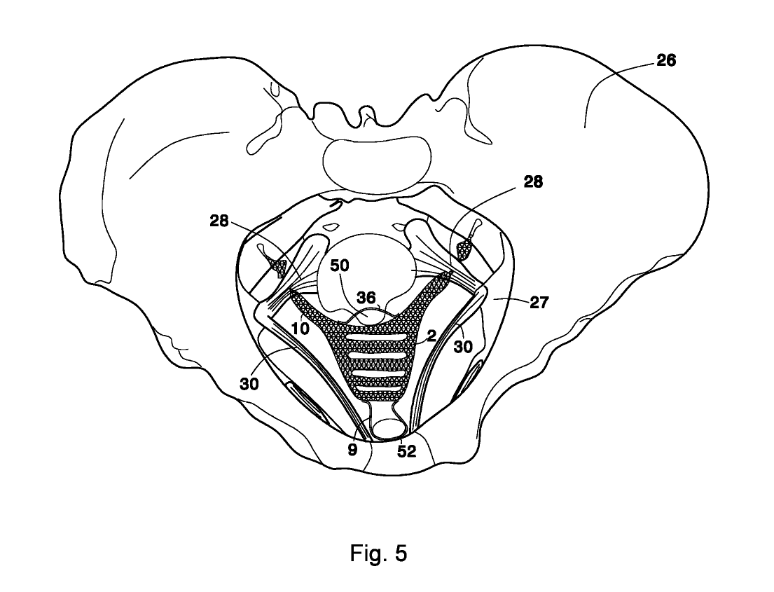

FIG. 5 illustrates the view of FIG. 2 after installing the posterior implant.

Posterior implant 2 is used for reconstructing the posterior pelvic floor, including prolapse of the colon, the small intestine and/or the uterus (womb).

Two straps 10 of posterior implant 2 are inserted into two SS ligaments 28.

Loop 36 may be sutured to cervix 50, and loop 9 may be sutured to perineal body 52 for improving the strength and security of the connection.

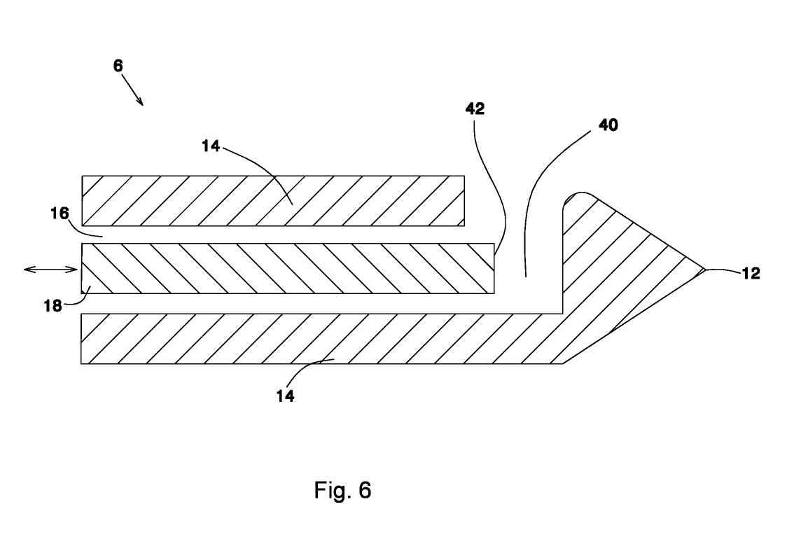

FIG. 6 illustrates the head of a needle for threading the straps of the implants, according to one embodiment of the present invention.

A needle 6 is used for threading each of straps 10 through ATFP ligaments 30 and SS ligaments 28.

The head of needle 6 includes a rod 18, which may be manually slid back and forth in a track 16 within a body 14, as in brake cables.

The edge 42 of rod 18 may be inserted into a niche 40. The tip 12 of needle 6 is located at the edge of body 14.

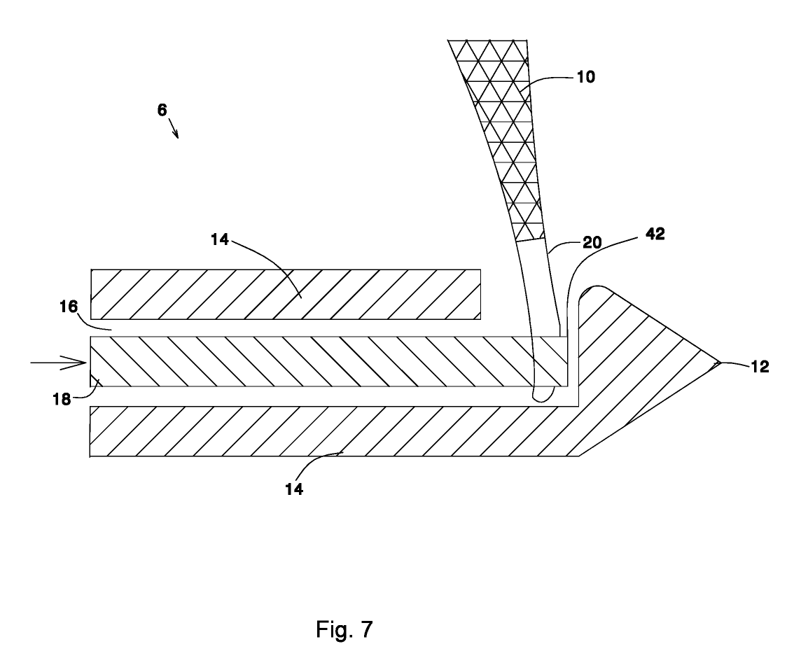

FIG. 7 illustrates the first step of threading the straps of the implants, using the needle of FIG. 6.

The surgeon inserts looped end 20 of strap 10 into niche 40, and traps it by rod edge 42 of rod 18 into looped end 20.

In case that the surgeon has not succeeded in inserting rod 18 into looped end 20, the surgeon may trap looped end 20 by applying physical force of rod edge 42 towards the limiting wall thereof in niche 40.

According to another embodiment the surgeon may trap strap 10 directly by applying physical force of rod edge 42 on the end of strap 10 towards the limiting wall thereof in niche 40.

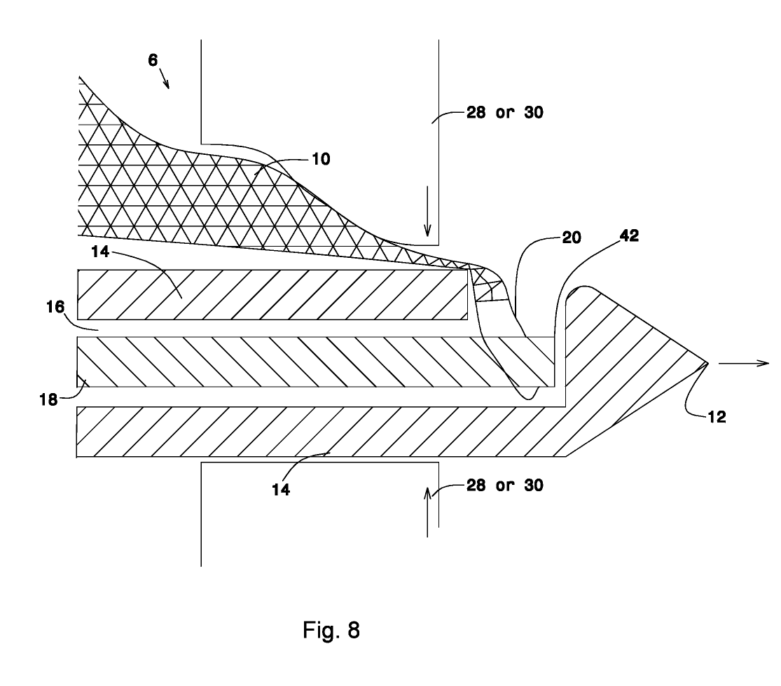

FIG. 8 illustrates the second step of threading the straps of the implants, using the needle of FIG. 6.

The surgeon then pushes tip 12 together with body 14 into an ATFP ligament 30 or into an SS ligament 28, threading looped end 20 and strap 10 through the ligament.

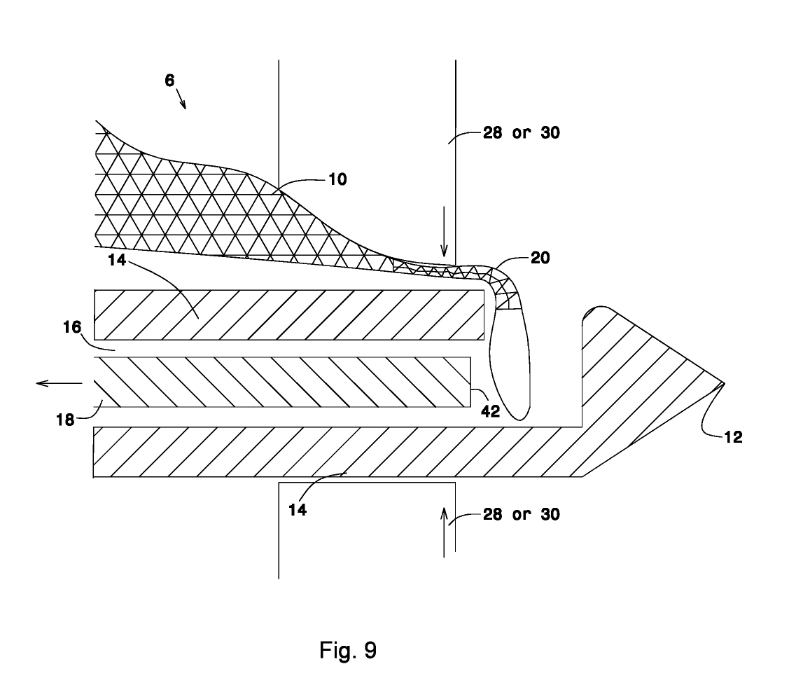

FIG. 9 illustrates the third step of threading the straps of the implants, using the needle of FIG. 6.

The surgeon then slides rod edge 42 out of niche 40, releasing looped end 20 from rod edge 42.

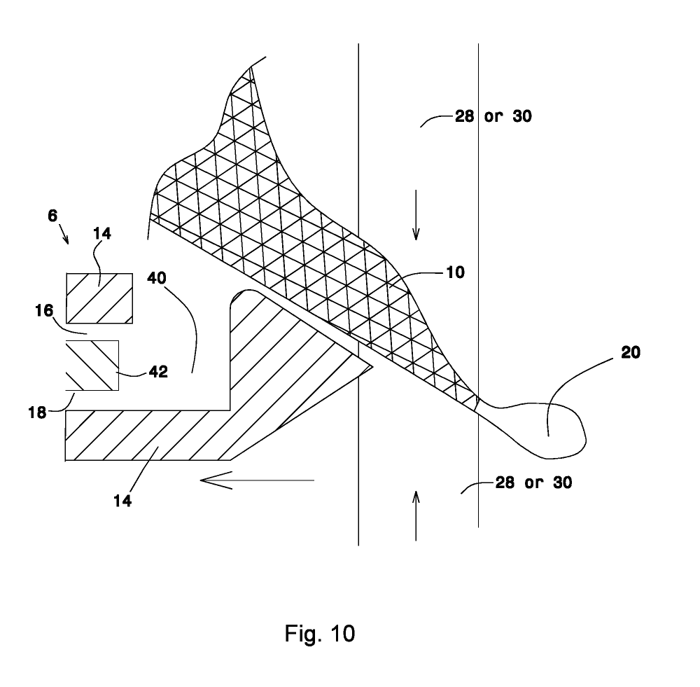

FIG. 10 illustrates the fourth step of threading the straps of the implants, using the needle of FIG. 6.

The surgeon then pulls body 14 together with tip 12 out of ATFP ligament 30 or SS ligament 28. Since looped end 20 has been released at the third step, and since ligament 30 (or 28) shrinks tightly, as shown by the arrows, strap 10 remains threaded while tip 12 exits.

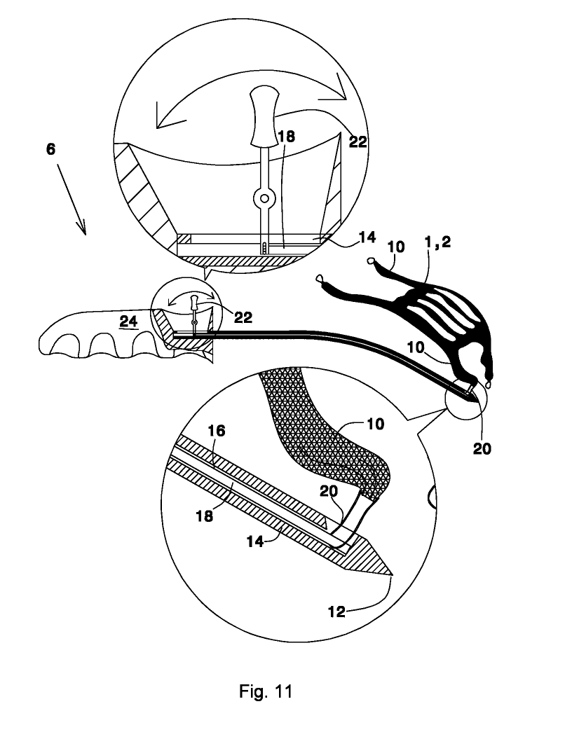

FIG. 11 illustrates the needle of FIG. 6 and its operation.

The surgeon holds handle 24 of needle 6, and slides rod 18 by toggling a toggle arm 22, which is connected to rod 18.

Needle 6 as a whole may be flexible like a brake cable, thin and long enough to occupy minimal surgery space.

Since tip 12 is inserted into the pelvic area, and toggle arm 22 is far away tip 12, toggle arm is located outside the body of the patient and may be located farther and outside the surgical area.

It may be appreciated according to these steps that the surgeon can thread strap 10 from the side having surgical access, without requiring any additional perforations of the body from the opposing direction.

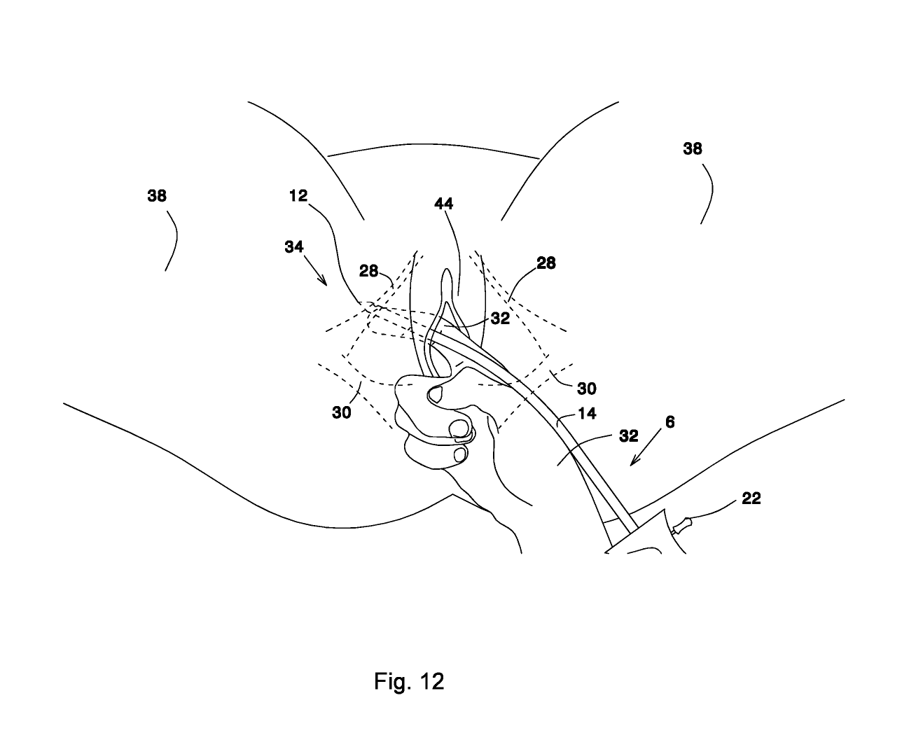

FIG. 12 illustrates the operation of the needle of FIG. 6 in aspect of the surgeon's access to the pelvic area.

The surgeon inserts finger 32 thereof into the vagina 44 between the patient's legs 38 and reaches pelvic area 34 (the lines of the parts inside are dashed). The surgeon then separates an SS ligament 28 from the other organs, locates tip 12 of needle 6 on a selected threading point on SS ligament 28, and traps trapping looped end 20 to niche 40 of needle 6.

The surgeon then penetrates tip 12 through SS ligament 28 and pushes into the desired depth; then releases looped end 20 from needle 6 by toggling toggle arm 22, using the other hand thereof.

The surgeon can then pull tip 12 back, leaving looped end 20 and strap 10 at the side beyond, having tight shrinking of SS ligament 28 towards strap 10 at the threaded point.

Tying of strap 10 is not required due to natural tying of SS ligament 28 to strap 10.

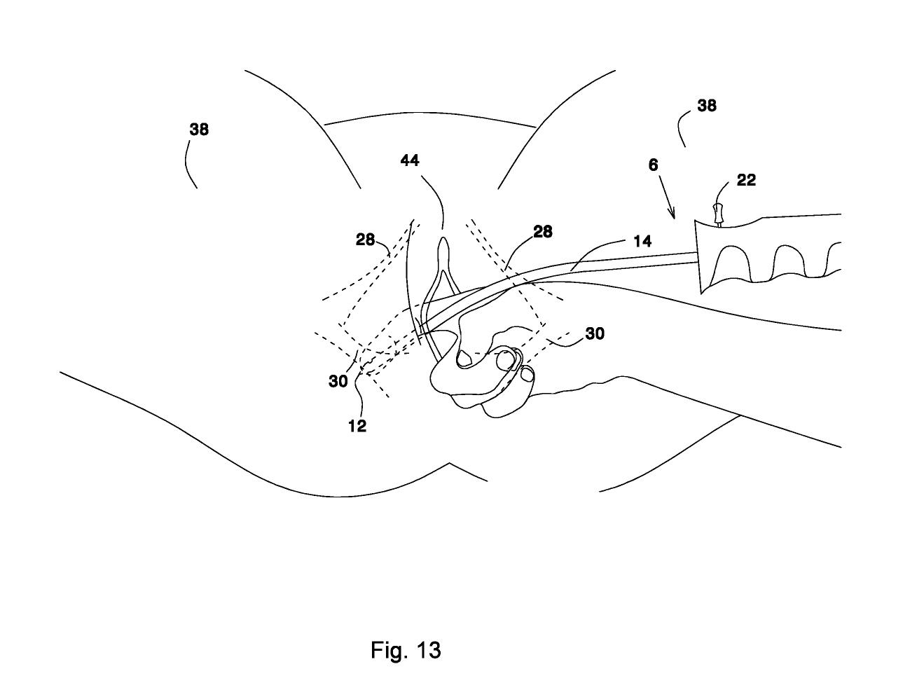

FIG. 13 illustrates the operation of the needle of FIG. 6 in aspect FIG. 12, to another ligament.

The surgeon inserts the finger 32 thereof into vagina 44, then separates an ATFP ligament 30, and locates tip 12 of needle 6 on the selected threading point, after trapping looped end 20 to niche 40 of needle 6.

The surgeon then penetrates tip 12 through ATFP ligament 30 and on to the desired depth; then releases looped end 20 from needle 6 by toggling toggle arm 22, using the other hand thereof, then pulls tip 12 back leaving looped end 20 and strap 10 at the side beyond, having tight shrinking of ATFP ligament 30 towards strap 10 at the threaded point.

In the figures and description herein, the following numerals and symbols have been mentioned:

numeral 1 denotes an anterior implant;

numeral 2 denotes a posterior implant;

numeral 4 denotes a space for reducing the weight of an implant;

numeral 6 denotes a needle according to one embodiment of the present invention;

numeral 8 denotes a loop in the anterior implant for anchoring it to the cervix;

numeral 9 denotes a loop in the posterior implant for anchoring it to the perineal body;

numeral 10 denotes a strap extending from the implant;

numeral 12 denotes a tip of the inventive needle;

numeral 14 denotes the body of the inventive needle;

numeral 16 denotes a track within the body of the needle;

numeral 18 denotes a rod traveling within the body of the needle;

numeral 20 denotes a looped end at the edge of the implant strap;

numeral 22 denotes a toggle arm for trapping and releasing the looped end;

numeral 24 denotes a handle of the needle;

numeral 26 denotes the ischium (bone);

numeral 27 denotes the ischial spine (bone);

numeral 28 denotes a sacrospinous (SS) ligament;

numeral 30 denotes an arcus tendineous fascia pelvic (ATFP) ligament;

numeral 32 denotes a surgeon's finger;

numeral 34 denotes the pelvic area;

numeral 36 denotes a loop in the posterior implant for anchoring it to the cervix;

numeral 38 denotes a patient's leg;

numeral 40 denotes a niche in the needle for trapping the looped end of the strap;

numeral 42 denotes the edge of the rod sliding in the track;

numeral 44 denotes the vagina, into which the surgeon inserts the finger thereof;

numeral 46 denotes the sacrum (bone);

numeral 48 denotes the uterus (womb);

numeral 50 denotes the cervix, extending from the uterus; and

numeral 52 denotes the perineal body;

While certain features of the invention have been illustrated and described herein, the invention can be embodied in other forms, ways, modifications, substitutions, canchores, equivalents, and so forth. The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A needle for surgical threading of a strap of an implant through a surface, said needle comprising:
a trap for trapping said strap to said needle while said needle is at the accessible side of said tissue;
a tip for threading said trapped strap from said accessible side of said tissue; and
a mechanism for releasing said trap, said mechanism driven from said accessible side of said tissue,
thereby allowing returning of said tip to said accessible side of said tissue while abandoning said strap at the threaded point, thus performing said threading from said accessible side of said tissue.

2. A needle according to claim 1, wherein said trap comprises:
a niche, for inserting an end of said strap of said implant; and
a rod, for grasping said end of said strap.

3. A needle according to claim 2, wherein said niche is located near said tip.

4. A needle according to claim 2, wherein said end of said strap comprises a looped end for inserting said rod thereinto.

5. A needle according to claim 2, wherein said rod is capable of applying physical force on said end of said strap towards the limiting wall thereof in said niche.

6. A needle according to claim 1, wherein said mechanism for releasing said trap is manually driven.

7. A needle according to claim 2, wherein said mechanism for releasing said trap comprises a cable, driven from said accessible side of said tissue, for removing said rod from said end of said strap.

8. A needle according to claim 1, further comprising an arm for driving said mechanism, said arm located outside the surgical area.

9. An anterior implant comprising:
at least two first straps for threading thereof into the arcus tendineous fascia pelvic (ATFP) ligaments;
at least two second straps for threading thereof into the sacrospinous (SS) ligaments; and
a loop between said second straps for anchoring thereof to the cervix.

10. An anterior implant according to claim 9, wherein said implant is used for reconstructing the organs selected from the group including: prolapse of the urinary bladder, the colon, the small intestine.

11. An anterior implant according to claim 9, further comprising spaces for reducing weight of said implant.

12. A posterior implant comprising:
at least two straps for threading thereof into the sacrospinous (SS) ligaments;
a first loop between said straps for anchoring thereof to the cervix; and
a second loop at the side opposing said straps, said second loop for anchoring thereof to the perineal body.

13. A posterior implant according to claim 12, wherein said implant is used for reconstructing the organs selected from the group including: the colon, the small intestine, the uterus.

14. A posterior implant according to claim 12 further comprising spaces for reducing weight of said implant.

15. A method for using a needle to thread a strap through a surface, said method comprising the steps of:
trapping an end of said strap while said needle is at the accessible side of the surface;
threading said needle together with said trapped strap through said surface, from said accessible side of said surface;
releasing said trap, such that driving of said releasing is from said accessible side; and
returning said needle to said accessible side while abandoning said strap at the threaded point,
thereby performing said threading from said accessible side of said surface.

16. A method for threading a strap according to claim 15, wherein said trapping of said end of said strap comprises the steps of:
inserting said end of said strap into a niche; and
grasping said end of said strap.

17. A method for threading a strap according to claim 16, wherein said grasping of said end of said strap comprises the step of inserting a rod of said needle into a looped end of said strap.

18. A method for threading a strap according to claim 16, wherein said grasping of said end of said strap comprises the step of applying physical force on said end of said strap towards the limiting wall thereof in said niche.

19. A method for threading a strap according to claim 16, wherein said releasing comprises removing said rod from said end of said strap.

20. A method for installing an anterior implant, said method comprising the steps of:
threading at least two first straps of said implant into the arcus tendineous fascia pelvic (ATFP) ligaments;
threading at least two second straps of said implant into the sacrospinous (SS) ligaments; and
anchoring a loop between said second straps to the cervix.

21. A method for installing a posterior implant, said method comprising the steps of:
threading at least two straps of said implant into the sacrospinous (SS) ligaments;
anchoring a first loop between said straps, to the cervix; and
anchoring a second loop at the side opposing said straps, to the perineal body.
